(19) United States
(12) Patent Application Publication
(10) Pub. No.: US 2026/0077189 A1
(43) Pub. Date: Mar. 19, 2026

(54) DEVICE AND SYSTEM FOR FACILITATING THE DIFFUSION OF A SUBSTANCE THROUGH BIOLOGICAL TISSUE

(71) Applicant: OgeewOw, LLC, Canonsburg, PA (US)

(72) Inventors: James Goldsmith, McMurray, PA (US); Brandon Novinc, Strongsville, OH (US)

(73) Assignee: OgeewOw, LLC, Canonsburg, PA (US)

(21) Appl. No.: 19/329,757

(22) Filed: Sep. 16, 2025

Related U.S. Application Data
(60) Provisional application No. 63/695,006, filed on Sep. 16, 2024.

(51) Int. Cl. A61N 1/30; A61M 5/24; A61M 5/315; A61M 35/00; A61N 1/04

(52) U.S. Cl. CPC A61N 1/303; A61M 5/2422; A61M 5/2466; A61M 5/31511; A61M 5/3158; A61M 35/006; A61N 1/044; A61N 1/0472; A61M 2005/2407; A61M 2005/2485; A61M 2205/3324; A61M 2205/3327; A61M 2205/50; A61M 2205/587

(57) ABSTRACT

An iontophoresis device includes an outer housing, and a swab subassembly slidingly received within the outer housing that includes an elongated rod, a swab, a conductor extending along the rod and coupled to the swab. The iontophoresis device also includes an additional device subassembly, such as a syringe subassembly, that is slidingly received within the outer housing. The syringe subassembly includes an elongated body for holding a substance, a needle, and a plunger.

### CROSS REFERENCE TO RELATED APPLICATIONS

This application is claims priority to U.S. Patent Application Ser. No. 63/695,006, filed on Sep. 16, 2024 and titled “System for Facilitating the Diffusion of a Substance, Such as an Anesthetic or Another Drug, Through Biological Tissue,” the disclosure of which is incorporated herein by reference.

### FIELD OF THE INVENTION

The disclosed concept relates generally to medical devices and procedures wherein a substance is delivered to a subject, and, in particular, to a device and system for facilitating the diffusion of a substance, such as an anesthetic or another drug, through the biological tissue of a subject.

### BACKGROUND OF THE INVENTION

A number of different anesthetic drugs are used during dental procedures. For example, for procedures like fillings and crowns, a local anesthetic drug is typically used.

Common local anesthetic drugs used in dentistry include lidocaine, articaine, prilocaine, mepivacaine, and bupivacaine. These drugs impart a numbing effect that lasts for 30 to 60 minutes that allows the dental procedure to be performed with little to no discomfort to the patient. Local anesthetic drugs may be administered topically, such as by being rubbed onto the patient's gum tissue, or as an injection into the part of the patient's mouth that requires treatment.

In addition, there are many other medical procedures that require the administration of drugs by injection. For example, and without limitation, Botox® and steroids are often administered by way of injections. The discomfort associated with such injections, for some patients, can be an obstacle that prevents them from deciding to go forward with the treatment. Also, for some drugs that are administered topically, their effectiveness may be limited due to the fact that the penetration of those drugs could be subject to limited absorption.

There is thus room for improvement in the field of drug delivery devices and procedures.

### SUMMARY OF THE INVENTION

In one embodiment, an iontophoresis device is provided that includes an outer housing, a swab subassembly structured and configured to be slidingly received within the outer housing, the swab subassembly including an elongated rod, a swab provided at a first end of the elongated rod, and a conductor extending along the rod and coupled to the swab, and a device subassembly, such as a syringe subassembly, a light subassembly, or a pH measuring subassembly, structured and configured to be slidingly received within the outer housing adjacent the swab subassembly. The swab subassembly is structured and configured to slide along the longitudinal axis of the outer housing in a manner that causes the swab to extend out of a first end of the outer housing and the device subassembly is also structured and configured to slide along the longitudinal axis of the outer housing.

In another embodiment, an iontophoresis device is provided that includes an outer housing having an outer wall, wherein the outer wall includes a first slot extending along a longitudinal axis of the outer housing and a second slot extending along the longitudinal axis of the outer housing, a swab subassembly slidingly received within the outer housing, the swab subassembly including an elongated rod, a swab provided at a first end of the elongated rod, a conductor extending along the rod and coupled to the swab, and a first tab member extending from an outer portion of the swab subassembly, wherein the first post member is received through the first slot, and a syringe subassembly slidingly received within the outer housing, the syringe subassembly having a vial for holding a substance, a needle coupled to a first end of the vial, a plunger received within the vial and structured to force the substance out of the vial and through the needle, and a second tab member extending from an outer portion of the syringe subassembly, wherein the second tab member is received through the second slot. Movement of the first tab member within the first slot will cause the swab subassembly to slide along the longitudinal axis of the outer housing and movement of the second tab member within the second slot will cause the syringe subassembly to slide along the longitudinal axis of the outer housing.

In another embodiment, an iontophoresis system is provided that includes a control unit structured and configured to generate an electrical current, and an iontophoresis device as just described. The conductor of the iontophoresis device is structured to be coupled to the control unit for receiving the electrical current. Responsive to the electrical current, a charge will be provided to a distal end of the conductor to provide an electro-repulsive force to a substance, such as an anesthetic or another drug. that is provided on or within the absorbent tip.

### BRIEF DESCRIPTION OF THE DRAWINGS

A full understanding of the invention can be gained from the following description of the preferred embodiments when read in conjunction with the accompanying drawings in which:

FIG. 1 is a schematic diagram of an iontophoresis system including an iontophoresis device for delivering a substance, such as an anesthetic, to a subject according to an exemplary embodiment of the disclosed concept;

FIG. 2 is an isometric view of the iontophoresis device of FIG. 1 that shows the syringe subassembly thereof in the extended position and the swab subassembly thereof in the retracted position;

FIG. 3 is an isometric view of the iontophoresis device of FIG. 1 that shows the syringe subassembly thereof in the retracted position and the swab subassembly thereof in the extended position;

FIG. 4 is an exploded view of the iontophoresis device of FIG. 1 showing the various components thereof;

FIG. 5 is a rear isometric view of the outer housing of the iontophoresis device of FIG. 1;

FIGS. 6 and 7 are front and rear isometric views, respectively, of a vial forming a part of the syringe subassembly of the iontophoresis device of FIG. 1;

FIG. 8 is an isometric view of a syringe tip forming a part of the syringe subassembly of the iontophoresis device of FIG. 1;

FIGS. 9 and 10 are isometric views illustrating the assembly of a portion of the swab subassembly of the iontophoresis device of FIG. 1;

FIG. 11 is an isometric view of a swab member forming a part of the swab subassembly of the iontophoresis device of FIG. 1;

FIG. 12 is a schematic diagram of an iontophoresis device according to an alternative exemplary embodiment of the disclosed concept.

### DETAILED DESCRIPTION OF THE INVENTION

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

Iontophoresis, or electromotive drug administration, is a process that enhances the delivery of drugs or other substances through a biological membrane via the application of low-intensity electrical current. Iontophoresis offers several advantages over oral and injection-based drug delivery, including the avoidance of pain and the potential for infection associated with a needle injection, the ability to control the rate of drug delivery, the ability to program the drug-delivery profile, and the minimization of local tissue trauma.

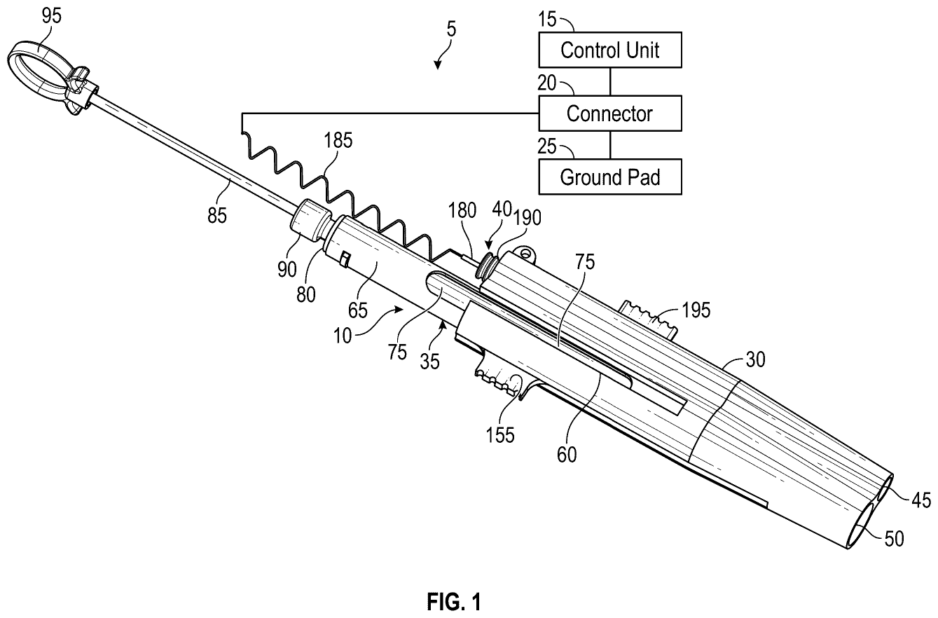

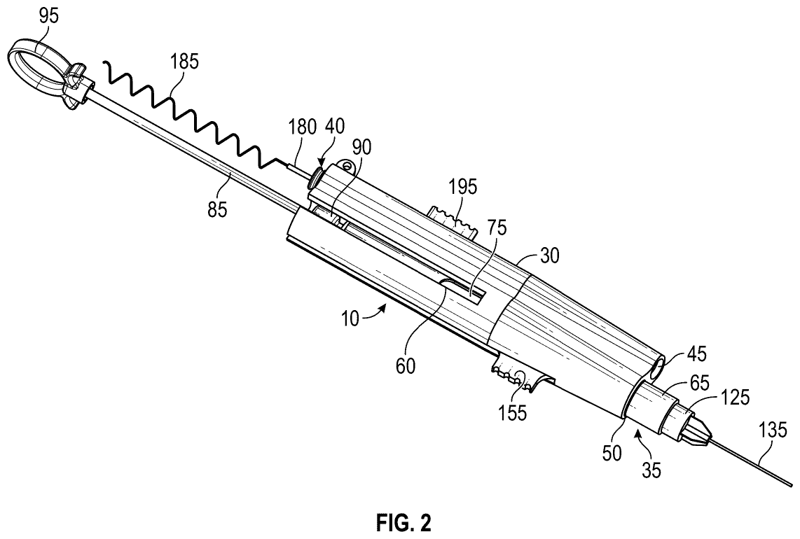

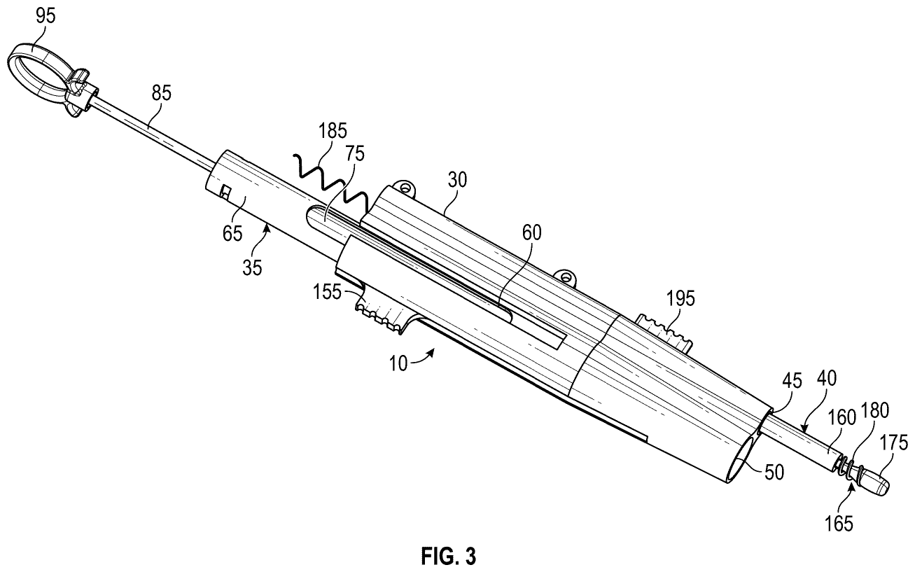

FIG. 1 is a schematic diagram of an iontophoresis system 5 for delivering a substance, such as, without limitation, an anesthetic for dental or other procedures or a drug such as Botox® or a steroid, to a subject according to a non-limiting exemplary embodiment of the disclosed concept. As seen in FIG. 1, iontophoresis system 5 includes an iontophoresis device 10, a control unit 15, a connector 20, and a ground pad 25, each of which is described in more detail herein. Iontophoresis device 10 includes an outer housing 30, a syringe subassembly 35 (described in detail herein) and a swab subassembly 40 (described in detail herein). As described herein, syringe subassembly 35 and swab subassembly 40 are received and held within outer housing in a manner that permits both syringe subassembly 35 and swab subassembly 40 to be independently slidable relative to outer housing. As a result, syringe subassembly 35 and swab subassembly 40 are each able to be moved between a retracted position and an extended position relative to outer housing 30. FIG. 2 is an isometric view of iontophoresis device 10 that shows syringe subassembly 35 in the extended position and swab subassembly 40 in the retracted position. FIG. 3 is an isometric view of iontophoresis device 10 that shows syringe subassembly 35 in the retracted position and swab subassembly 40 in the extended position.

Iontophoresis system 5 is structured and configured to allow the operator thereof, such as a dentist, registered dental hygienist, physician, physician's assistant, nurse or other medical professional, to diffuse a substance through a tissue of the subject, such as the subject's gums, by iontophoresis using swab subassembly 40 (described in detail herein). In addition, as described herein, iontophoresis system 5 is also structured and configured to allow the operator to inject the substance into the subject's tissue by way of syringe subassembly 35 (described in detail herein) without having to remove swab subassembly 40 from the application site. In the exemplary embodiment, such diffusion may be performed while keeping syringe subassembly 35 out of site. Syringe subassembly 35 can also be used to refresh the substance on swab subassembly 40 by releasing the substance onto swab subassembly 40, including while it remains in contact with the application site.

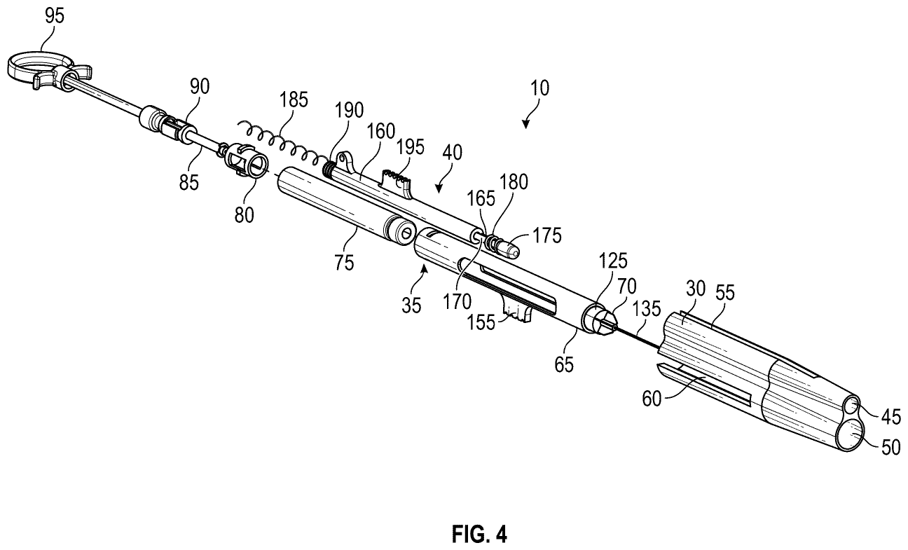

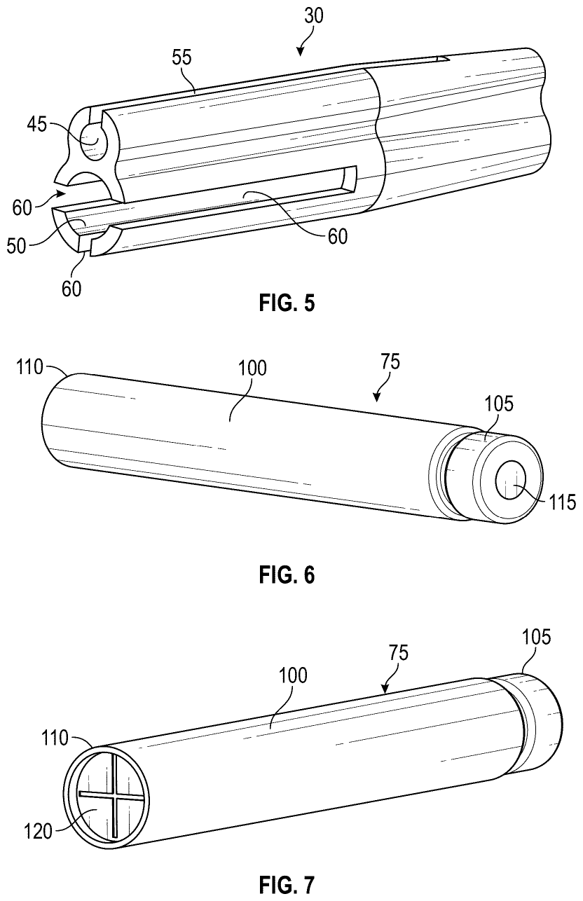

FIG. 4 is an exploded view of iontophoresis device 10 showing the various components thereof. As noted above, iontophoresis device 10 includes outer housing 30 for slidably holding syringe subassembly 35 and swab subassembly 40. FIG. 5 is a rear isometric view of outer housing 30. As seen in FIGS. 4 and 5, outer housing 30 in the exemplary embodiment has a dual tube configuration that includes a first tube portion 45 structured for receiving swab subassembly 40 and a second tube portion 50 structured for receiving syringe subassembly 35. First tube portion 45 includes a slit 55 provided in the outer wall thereof that extends along the longitudinal axis of first tube portion 45, and second tube portion 50 includes slits 60 provided in the outer wall thereof that extend along the longitudinal axis of second tube portion 50. Outer housing 30 may be made of a rigid polymer material.

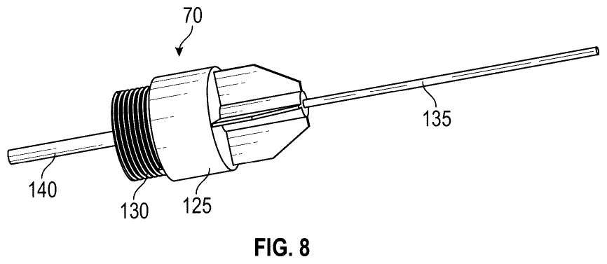

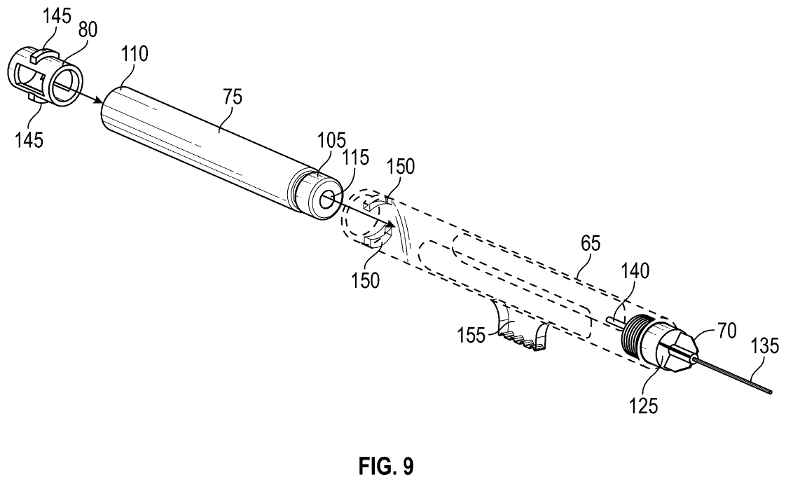

Referring to FIG. 4, syringe subassembly 35 includes a syringe housing 65, a syringe tip 70, a vial 75 for holding the substance (e.g., a local anesthetic such as lidocaine, or another substance such as Botox® or a steroid) to be delivered by iontophoresis system 5, a vial retainer 80, a push rod 85, an inner plug retainer 90 and a syringe pusher head 95. FIGS. 6 and 7 are front and rear isometric views, respectively, of vial 75. Vial 75 includes a main body 100 having a front end 105 and a rear end 110. A flexible membrane 115, such as a resealable rubber, is provided at front end 105. A slidable sealing disk 120 is provided at rear end 110. FIG. 8 is an isometric view of syringe tip 70. Syringe tip 70 includes a base member 125 having thread 130. A first needle 135 extends from the front end of base member 125 and a second needle 140 extends from the rear end of base member 125.

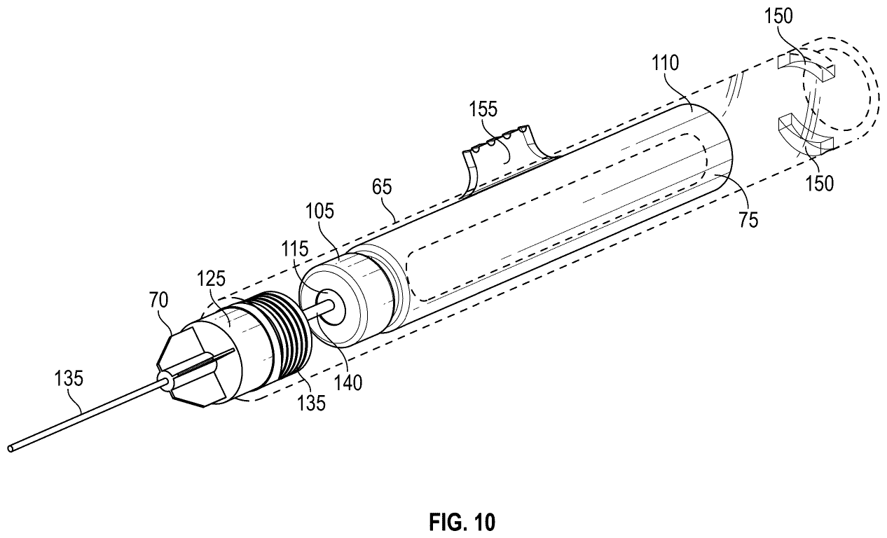

A shown in FIGS. 4, 9 and 10, syringe tip 70 is attached to the front end of syringe housing 65 by a threaded connection. As illustrated in FIGS. 9 and 10, vial 75 is inserted into the interior of syringe housing 65 to a point where second needle 140 punctures flexible membrane 115, and thereafter vial retainer 80 is inserted in the rear end of syringe housing 65 behind vial 75. As seen in FIG. 9, vial retainer 80 includes external tabs 145 and the interior of syringe housing 65 includes internal tabs 150. External tabs 145 are structured to engage internal tabs 150 to prevent vial retainer 80 from sliding too far inside syringe housing 65. As will be appreciated, once inserted, vial retainer 80 prevents vial 75 from sliding out of syringe housing 65. In an alternative embodiment, a harpoon hook like tip could be provided at the end of push rod 85 that engages into the distal end of vial 75 where the seal is located to aid in aspiration of the therapeutic substance and to help to discern whether needle 135 has penetrated into a blood vessel.

In addition, referring to FIGS. 1 and 4, after vial 75 and vial retainer 80 are inserted into syringe housing 65 as just described, the proximal end of push rod 85 and inner plug retainer 90 are inserted into syringe housing 65 through vial retainer 80. As a result, the proximal end of push rod 85 will be adjacent to and in engagement with slidable sealing disk 120 so that push rod 85 and slidable sealing disk 120 together form a plunger. Thus, if a user engages syringe pusher head 95 and pushes push rod 85 along the longitudinal axis of syringe housing 65, slidable sealing disk 120 will be caused to slide within main body 100 of vial 75 and cause the substance contained therein to be ejected out of first needle 135. In addition, the so assembled syringe subassembly 35 is then inserted into second tube portion 50 of outer housing 30. In particular, syringe housing 65 includes a tab member 155 that is received within one of the slots 60. As a result, syringe subassembly 35 may be slid along the longitudinal axis of second tube portion 50 of outer housing 30 by exerting a force on tab member 155. In the extended position shown in FIG. 2, this will cause first needle 135 to extend out of the end of outer housing 30.

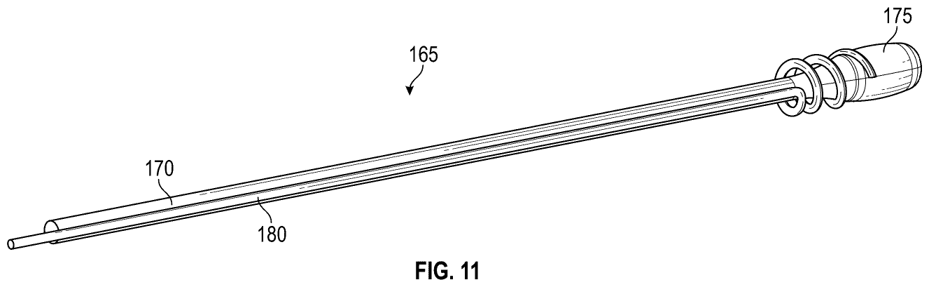

Referring again to FIG. 4, swab subassembly 40 includes a swab housing 160 that houses therein a swab member 165. Swab member 165 is shown in FIG. 11 and includes a swab rod 170 having a swab 175 attached to the front end thereof. Swab 175 may be made of any suitable absorbent material, such as, without limitation, a fiber material such as cotton or a foam material. Swab 175 and a portion of swab rod 170 extend out of the front end of swab housing 160. A conductive wire 180 extends along the length of swab rod 170 and coils about the front end thereof, including around and in contact with a portion of swab 175. A wire 185 is connected to the end of wire 180 that is opposite swab 175. As seen in FIG. 1, wire 185 is connected to connector 20. In addition, a grommet 190 closes of the rear end of swab housing 160, and wires 180 extends through grommet 190. The assembled swab subassembly 40 is inserted into first tube portion 45 of outer housing 30. In particular, swab housing 160 includes a tab member 195 that is received within slot 55. As a result, swab subassembly 40 may be slid along the longitudinal axis of first tube portion 45 of outer housing 30 by exerting a force on tab member 195. In the extended position shown in FIG. 3, this will cause swab 175 and a portion of swab housing 160 to extend out of the end of outer housing 30.

Moreover, control unit 15 is structured and configured to generate an electrical current, by way of an appropriate power source, which is then provided to wires 185 and 180 through connector 20. As a result, a charge/voltage (positive or negative) is provided at the distal end of wire 180. It is this charge/voltage that enables the iontophoresis to occur. In particular, when a charged substance (with the same polarity as the charge on the wire), such as an anesthetic or other drug, is provided on and/or within swab 175, such as by dipping swab 175 into the substance or causing the substance to be ejected from first needle 135 and onto swab 175 as described herein, and the electrical current is provided by control unit 15, the electrical current will provide an electro-repulsive force to the substance that drives the substance away from swab 175 and into the biological membrane that is in contact with swab 175 since both the distal end of wire 180 and the substance have the same polarity charge.

In addition, as described above, in the exemplary embodiment, ground pad 25 is also coupled to connector 20. Ground pad 25 is in the form of a patch that is configured to engage exposed skin of the subject (or another grounding surface) to provide an electrical ground return path. As a result, the distal end of wire 180 serves as an anode and the subject's skin (or another grounding surface) serves as a cathode due to engagement with ground pad 20. Thus, in the non-limiting case where the substance is an anesthetic used in a dental procedure, the electro-repulsive force provided through wire 180 drives the anesthetic into the subject's gum tissue, thereby anesthetizing the gum tissue. Similarly, in other medical procedures, such as where Botox® or a steroid is administered, the electro-repulsive force provided through wire 180 drives the drug into the subject's skin or other tissue. The electrical current may be applied for controlled amounts of time in order to control the total amount of charge, and the level and duration of the electro-repulsive force.

In one exemplary embodiment, control unit 15 may be a free-standing device that is separate from iontophoresis device 10. In this embodiment, control unit 15 may be structured and configured to be connected to a standard wall outlet for receiving the power that is necessary to generate the electrical current that is provided to iontophoresis device 10. Alternatively, in another exemplary embodiment, control unit 15 maybe integrated within iontophoresis device 10 such that iontophoresis device 10, control unit 15, and ground pad 25 form an integrated unit. For example, in this embodiment, control unit 15 may be provided within outer hosing 30 acting as a housing. In addition, in this embodiment, control unit 15 may be provided with the electrical current generating circuit and an integrated source of power, such as a rechargeable battery. In this way, this embodiment of iontophoresis device 10 would be able to be used as an integrated, handheld device without the need to separately connect to a separate, free standing device for operation. As such, this embodiment would be particularly useful during dental or medical procedures as it would essentially be a “wireless”free-standing integrated device.

Thus, iontophoresis system 5 including iontophoresis device 10 described herein in the various exemplary embodiments is structured and configured to allow the operator, such as a dentist, registered dental hygienist, physician, physician's assistant, nurse or other medical professional, to diffuse a substance through a subject's tissue using swab subassembly 40 as described herein. Diffusing the substance using swab subassembly 40 may enable the substance to be diffused deeper into the tissue as compared to simply applying it topically, and may enable less of the substance to be used while achieving the same therapeutic result. Iontophoresis device 10 as described herein is also structured and configured to allow the operator to inject the substance into the subject's tissue using syringe subassembly 35, with or without having to remove swab subassembly 40 from the application site. This is possible because both swab subassembly 40 and syringe assembly 35 may be selectively and separately slid within outer housing 30 using tabs 155 and 195. In addition, in the exemplary embodiment, syringe subassembly 35 may be used for injection of the substance while keeping push rod 85 and syringe pusher head 95 out of site of the subject. Also, syringe subassembly 35 may be used to refresh the substance on swab 175 by extending both syringe subassembly 35 and syringe assembly 35 to the forward extended position and releasing the substance onto the swab 175, including when swab 175 is in contact with the subject's tissue (the application site).

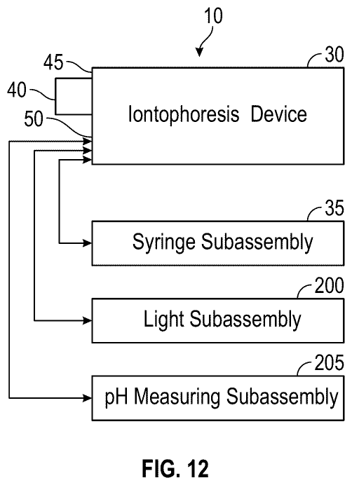

In a further alternative embodiment, syringe subassembly 35 may be removed from housing 30 and replaced with another, alternative device subassembly. The alternative device subassembly may be, for example and without limitation, a device subassembly that includes a light for illuminating the patient's tissue while the substance in question is being provided by iontophoresis as described herein or a device subassembly that includes a pH measuring device for measuring the pH level of the patient's tissue before, during and/or after the substance in question is being provided by iontophoresis as described herein. For example, the pH measuring device may comprise a pH glass or solid state microelectrode, or a fiber-optic pH sensor that uses optical fibers coated with pH-sensitive fluorescent or colorimetric dyes wherein the light signal changes depending on the pH. This is shown schematically FIG. 12, wherein a light subassembly 200 and/or a pH measuring subassembly 205 may be selectively inserted into hosing 30 within second tube portion 50. Light subassembly 200 and/or a pH measuring subassembly 205 may include a sub-housing similar to syringe housing 65 including tab member 155.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof.

What is claimed is:

1. An iontophoresis device, comprising:
an outer housing;
a swab subassembly structured and configured to be slidingly received within the outer housing, the swab subassembly including an elongated rod, a swab provided at a first end of the elongated rod, and a conductor extending along the rod and coupled to the swab; and
a device subassembly structured and configured to be slidingly received within the outer housing adjacent the swab subassembly;
wherein the swab subassembly is structured and configured to slide along the longitudinal axis of the outer housing in a manner that causes the swab to extend out of a first end of the outer housing and wherein the device subassembly is structured and configured to slide along the longitudinal axis of the outer housing.

2. The iontophoresis device according to claim 1, wherein the outer wall includes a first slot extending along a longitudinal axis of the outer housing and a second slot extending along the longitudinal axis of the outer housing, wherein the swab subassembly includes a first tab member extending from an outer portion of the swab subassembly, wherein the first tab member is structured and configured to be received through the first slot such that movement of the first tab member within the first slot will cause the swab subassembly to slide along the longitudinal axis of the outer housing, wherein the device subassembly includes a second tab member extending from an outer portion of the device subassembly, wherein the second tab member is structured and configured to be received through the second slot such that movement of the second tab member within the second slot will cause the device subassembly to slide along the longitudinal axis of the outer housing.

3. The iontophoresis device according to claim 2, wherein the device subassembly comprises a syringe subassembly, the syringe subassembly having a vial for holding a substance, a needle coupled to a first end of the vial, and a plunger received within the vial and structured to force the substance out of the vial and through the needle.

4. The iontophoresis device according to claim 3, wherein the outer housing has a first tube portion structured for receiving the swab subassembly and a second tube portion structured for receiving the syringe subassembly.

5. The iontophoresis device according to claim 3, wherein the first slot is provided in the first tube portion and the second slot is provided in the second tube portion.

6. The iontophoresis device according to claim 3, wherein the first tab member is perpendicular to a longitudinal axis of the swab subassembly and the second tab member is perpendicular to a longitudinal axis of the syringe subassembly.

7. The iontophoresis device according to claim 4, wherein the swab subassembly includes a swab housing, wherein the elongated rod and the conductor are provided at least partially within the swab housing, wherein the first tab member is provided on an outer surface of the swab housing, and wherein the swab housing is slidably received within the first tube portion.

8. The iontophoresis device according to claim 4, wherein the syringe subassembly includes a syringe housing, wherein the vial is provided within the syringe housing, wherein the second tab member is provided on an outer surface of the syringe housing, and wherein the syringe housing is slidably received within the second tube portion.

9. The iontophoresis device according to claim 8, wherein the vial includes a slidable sealing member provided within an interior of the vial, wherein the syringe subassembly includes a push rod structured to be at least partially receiving within the syringe housing and to engage the slidable sealing member, and wherein the push rod and the slidable sealing member form at least part of the plunger.

10. The iontophoresis device according to claim 9, wherein the syringe subassembly includes a pusher head coupled to the push rod.

11. The iontophoresis device according to claim 8, wherein the syringe subassembly includes a vial retainer structured to be received within the syringe housing behind the vial for retaining the vial within the syringe housing.

12. The iontophoresis device according to claim 11, wherein the syringe housing includes internal tabs, wherein the vial retainer includes external tabs, and wherein the external tabs are structured to engage the internal tabs when the vial retainer is inserted into the syringe housing.

13. The iontophoresis device according to claim 8, wherein the vial includes a flexible member at an end thereof, wherein the needle is provided a part of a syringe tip having a base, and a second needle connected to the base opposite the needle, wherein the second needle is in fluid communication with the needle, wherein the syringe tip is structured to be coupled to a first end of the syringe housing such that the second needle will be positioned to puncture the flexible member of the vial as the vial is moved toward the syringe tip.

14. The iontophoresis device according to claim 3, wherein the swab comprises a fiber material.

15. The iontophoresis device according to claim 14, wherein the fiber material comprises cotton.

16. The iontophoresis device according to claim 3, wherein the swab comprises a foam material.

17. The iontophoresis device according to claim 3, wherein a distal end of the conductor is coiled around the swab.

18. The iontophoresis device according to claim 3, further comprising a control unit structured and configured to generate an electrical current and provide the electrical current to the conductor, wherein the control unit is provided within the outer housing.

19. The iontophoresis device according to claim 1, wherein the device subassembly comprises a syringe subassembly, the syringe subassembly having a vial for holding a substance, a needle coupled to a first end of the vial, and a plunger received within the vial and structured to force the substance out of the vial and through the needle.

20. The iontophoresis device according to claim 19, further comprising a light subassembly, wherein the syringe subassembly is structured and configured to be selectively removable from the outer housing, and wherein the light subassembly is structured and configured to be inserted into the outer housing in place of the syringe subassembly.

21. The iontophoresis device according to claim 19, further comprising a pH measuring subassembly, wherein the syringe subassembly is structured and configured to be selectively removable from the outer housing, and wherein the pH measuring subassembly is structured and configured to be inserted into the outer housing in place of the syringe subassembly.

22. The iontophoresis device according to claim 1, wherein the device subassembly comprises a light subassembly.

23. The iontophoresis device according to claim 1, wherein the device subassembly comprises a pH measuring subassembly.

24. An iontophoresis system, comprising:
a control unit structured and configured to generate an electrical current; and
an iontophoresis device according to claim 1, wherein the conductor of the iontophoresis device is structured to be coupled to the control unit for receiving the electrical current, and wherein responsive to the electrical current a charge will be provided to a distal end of the conductor to provide an electro-repulsive force to a substance provided on or within the swab.

25. The iontophoresis system according to claim 24, further comprising a ground pad for providing an electrical ground return path for the electrical current.

26. The iontophoresis system according to claim 24, further comprising a connector, wherein the proximal end of the conductor and the ground pad are both coupled to the connector.

27. An iontophoresis system, comprising:
a control unit structured and configured to generate an electrical current; and
an iontophoresis device according to claim 3, wherein the conductor of the iontophoresis device is structured to be coupled to the control unit for receiving the electrical current, and wherein responsive to the electrical current a charge will be provided to a distal end of the conductor to provide an electro-repulsive force to a substance provided on or within the swab.

28. The iontophoresis system according to claim 27, further comprising a ground pad for providing an electrical ground return path for the electrical current.

29. The iontophoresis system according to claim 27, further comprising a connector, wherein the proximal end of the conductor and the ground pad are both coupled to the connector.
